(19) United States
(12) Patent Application Publication
(10) Pub. No.: US 2012/0208291 A1
(43) Pub. Date: Aug. 16, 2012

(54) METHODS AND COMPOSITIONS FOR MEASURING HIGH AFFINITY INTERACTIONS WITH KINETIC IMAGING OF SINGLE MOLECULE INTERACTION (KISMI)

(75) Inventors: M. Wayne Davis, Salt Lake City, UT (US); Erik M. Jorgensen, Salt Lake City, UT (US); Joel M. Harris, Salt Lake City, UT (US); Christopher E. Hopkins, Salt Lake City, UT (US); Joshua R. Wayment, Salt Lake City, UT (US); Eric Peterson, Salt Lake City, UT (US); Douglas Michael Kriech, Salt Lake City, UT (US)

(73) Assignee: Univeristy of Utah Research Foundation, Salt Lake City, UT (US)

(21) Appl. No.: 13/318,371

(22) PCT Filed: Apr. 30, 2010
(86) PCT No.: PCT/US10/33169

§ 371 Date: Apr. 23, 2012

Related U.S. Application Data
(60) Provisional application No. 61/226,799, filed on Jul. 20, 2009.
(60) Provisional application No. 61/215,066, filed on May 1, 2009.

(51) Int. Cl. G01N 21/64; C07K 17/00

(52) U.S. Cl. 436/501; 530/408; 530/300; 530/345

(57) ABSTRACT

Disclosed herein are methods and compositions relating to the detection and measuring of kinetic binding interactions.

This application claims the benefit of U.S. Provisional Application No. 61/215,066, filed May 1, 2009 and U.S. Provisional Application No. 61/226,799, filed on Jul. 20, 2009, which are incorporated by reference herein in their entirety.

### BACKGROUND OF THE INVENTION

Protein-protein interactions are critical for every aspect of biology. These interactions govern cell signaling, life-cycle regulation, biosynthetic pathways, the immunological response, control of gene expression, and vesicle-membrane fusions. Measuring the affinities of these interactions and their kinetics of binding and unbinding, is critical to understanding biology at the molecular level and to designing new pharmaceuticals. In particular, drugs with high affinity to their receptors (stronger than nM) are desirable for use as pharmaceuticals. For instance, monoclonal antibodies with high affinity to their targets are effective for treating cancer and they are lucrative for pharmaceutical companies. Methods that accurately measure the binding kinetics of high affinity interactions are useful in identifying drugs with the highest affinities from large collections of potential lead compounds or antibodies with sub-therapeutic affinities. Unfortunately, the methods presently commercially available are unable to rapidly and accurately measure high affinity interactions. Moreover, the currently commercially available technologies for rapid detection rely on high surface density display of target receptors to function properly and are thus unable to measure at the single molecule level.

### SUMMARY

Disclosed herein are methods and compositions for rapidly measuring the kinetics of high affinity interactions. Also disclosed are methods and compositions for measuring kinetics of high affinity interactions between single molecules.

In a further respect, the disclosed pertain to immobilizing protein thioesters on optically transparent surfaces in uniform orientations. In still a further aspect, the disclosed pertain to immobilizing protein thioesters on optically transparent surfaces in uniform orientations and as single molecules at optically-separated loci.

### BRIEF DESCRIPTION OF THE DRAWINGS

FIG. 1 shows the immobilization of cysteine onto the amine terminus of a PEG₅₀₀₀ diluted into a cyano passivated surface, along with the immobilization of a thioester protein to the cysteine.

FIG. 2 shows a Gray scale fluorescence image from tetramethylrhodamine immobilized to amine PEG tethers on glass, self assembled from a solution of 1.2 pM amine PEG₅₀₀₀ silane and 15 mM CETES. The circles represent located immobilized TMR molecules. The gray scale to the right shows the threshold set at 36 photoelectrons for locating TMR molecules.

FIG. 3 shows Gray scale fluorescence image from GFP labeled synaptobrevin immobilized to cysteine PEG tethers on glass. The circles represent located immobilized GFP labeled synaptobrevin. The gray scale to the right shows the threshold set at 59 photoelectrons used to locate GFP labeled synaptobrevin.

FIG. 4 shows the steps to effectuate slide modification: step 1: amination; step 2: addition of mPEG/NH₂—PEG; step 3: backfill surface amines; and step 4: addition of NH₂—PEG tether.

FIG. 5 shows a diagram of an amidated glass surface with a PEG monolayer and tethers extending into solution.

FIG. 6 shows a cartoon of a protein immobilized on a glass surface by a stable covalent peptide bond.

FIG. 7 shows the accumulation isotherm: anti-syntaxin Oyster 550 measured against immobilized Syntaxin.

### DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

Existing technologies for imaging and measuring the binding properties of a molecule have been limiting in that they are either indirect competition binding experiments, require dual fluorophore labeling in FRET-TIRF experiments where photobleaching events of labeled receptor limit data collection time, or are methods requiring high surface density of immobilized receptor which prevents direct measurement of binding interactions. Moreover, the previously existing technologies are unable to measure single molecule interactions. In order to overcome the limitations of existing technologies a new method capable of rapidly measuring the kinetics of high affinity interactions was developed and is referred to herein as Kinetic Imaging of Single Molecule Interactions (KISMI). Thus, disclosed herein, in one aspect, are methods of determining the kinetic binding properties of a first molecule (ligand) to a second molecule (receptor), comprising contacting one or more receptors with the first molecule and measuring the binding affinity interaction of the molecule, wherein the one or more receptors are bound to the surface of a substrate. Also disclosed are methods wherein the concentration of the receptor on the surface of the substrate is the same as or less than the concentration in bulk solution. Thus, for example, disclosed herein are methods wherein the surface concentration of the receptor is 10 times, 100 times, 1000 times, 10,000 times, 100,000 times, 10⁶ times, 10⁷ times, 10⁸ times 10⁹ times, 10¹⁰ times, 10¹¹ times, or 10¹² times less than the concentration of the bulk solution. It is further understood that by utilizing the methods disclosed herein measurements at the single molecule level can be performed at conditions where diffusion limitations are minimized to levels that do not alter the observed kinetic parameters.

It is understood that herein that the methods described herein can utilize receptors bound to a substrate. Thus, also disclosed herein are substrates with receptors bound to their surface. It is further disclosed that any of the disclosed compositions comprising a substrate and a receptor or methods of attaching a receptor to a substrate can be used in the methods of measuring binding interactions disclosed herein.

The term “alkyl” as used herein is a branched or unbranched saturated hydrocarbon group of 1 to 30 carbon atoms, for example, 3 to 30 carbon atoms, 1 to 20 carbon atoms, 1 to 18 carbon atoms, 1 to 14 carbon atoms. Examples of alkyl include, but are not limited to methyl, ethyl, n propyl, isopropyl, n butyl, isobutyl, t butyl, pentyl, hexyl, heptyl, octyl, decyl, tetradecyl, hexadecyl, eicosyl, tetracosyl and the like. The alkyl group can also be substituted or unsubstituted. The alkyl group can be substituted with one or more groups including, but not limited to, alkyl, halogenated alkyl, alkoxy, alkenyl, alkynyl, aryl, heteroaryl, aldehyde, amino, carboxylic acid, ester, halide, hydroxamate, hydroxy, ketone, nitro, silyl, sulfo-oxo, sulfonyl, sulfone, sulfoxide, or thiol, as described below. The term “halogenated alkyl” specifically refers to an alkyl group that is substituted with one or more halide, e.g., fluorine, chlorine, bromine, or iodine.

As used herein, the term “substituted” refers to all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described hereinbelow. The permissible substituents can be one or more and the same or different for appropriate organic compounds. For purposes of this disclosure, the heteroatoms such as nitrogen can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

A “cysteine residue,” as used herein, refers to a chemical residue that includes the general structure:

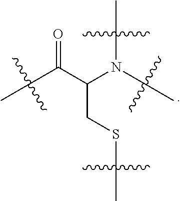

The D- or L-form of cysteine can be present in the cysteine residue.

### Substrates

The substrate can be any substrate suitable for chemical derivatization. The substrate can comprise silica, silicone wafer, borosilicate, soda-lime, quartz, gold, silver, platinum, or glass. In another aspect, the substrate can comprise a polymer, such as polydimethylsiloxane (PDMS). In a further aspect, the substrate can comprise or be coated with a metal suitable for chemical derivatization, such as gold or titanium. In one aspect, the substrate comprises glass. In another aspect, the substrate comprises silicon oxide.

The surface tether is bonded to a surface of the substrate, preferably through a covalent bond. Depending on the substrate used, a variety of chemical bonds can be present between the substrate surface and the surface tether. For example, when a gold substrate is used, a thiol bond between the gold surface and the surface tether can be present. In this example, the surface tether can be terminated at one or more ends with a thiol. In another example, when a glass substrate is used, a silane bond between the glass surface and the surface tether can be present. In this example, surface hydroxyl groups on the glass substrate can be reacted with a silane group of a surface tether to form a silane bond between the surface and the surface tether, as is further described below.

The surface tether functions to space the receptor from the surface. The surface tether can also ensure the desired orientation of the receptor relative to the surface. The attachment point of the receptor to the surface tether is preferably a cysteine residue or a suitable derivative thereof such as a hydrazine moiety. The cysteine residue or hydrazine is extended from the surface through a spacer group covalently bonded to the surface.

Tethering molecules contain a nucleophile reactive group at one end. Typically, for amines, the nuclephile reactive group is NHS but reactive groups can be used such as maleimides for thiol-derivitized surfaces, or other eletrophilic centers specific to surface nucleophile. The spacer group of the surface tether can comprise a number of residues that are suitable for surface derivatization. Preferably, the spacer group comprises a substituted or unsubstituted C₃-C₃₀ alkyl group, a polyethylene glycol (PEG) group, long-chain alkyls, aryls, esters, polynucleotides, poly-dextrans (or carbohydrates), or a peptide linker. Examples of C₃-C₃₀ alkyl groups include without limitation substituted or unsubstituted propyl, hexyl, octyl, decyl, C₁₂, C₁₄, C₁₆, C₁₈, C₂₀, C₂₄, C₂₆, C₂₈, or C₃₀. The polyethylene glycol (PEG) group can have a molecular weight of from about 300 to about 10,000,000 Daltons. More specifically, the PEG group can have a molecular weight of from about 2,000 to 20,000 Daltons, for example 2,000 Daltons or 5,000 Daltons. The peptide linker can comprise one or more suitable amino acids, for example, from about 1 to about 100 amino acids, including peptide linkers comprising 5, 8, 10, 15, or 18 amino acid residues.

At least one end of the surface tether comprises a moiety that can be reacted with the surface of the substrate. As discussed above, this group can vary depending on the substrate used. For example, when the substrate is glass, the surface tether can comprise a silane group that will react with surface hydroxyls to form a silane bond between the surface and the surface tether. Thus, the above disclosed surface tethers can comprise a terminal silane. In one specific example, the surface tether comprises a silane-PEG, such as a silane-PEG₅₀₀₀ (a PEG having a molecular weight of about 5,000 Daltons). At the other end of the tethers the there are two types of termini. Some tethers have unreactive groups such as a methoxy group, but other inert termini could be used such as hydroxyl, ethoxyl, and propyloxyl. The other type of tether termini is a protected nucleophile. Typically it is an amine with FMOC protective groups, but other nucleophiles, such as thiols and azides could be used. Protection of nucleophile is typically FMOC but could be substituted with any of the peptide chemistry protecting groups such as t-BOC, allyloxycarbonyl and benzyloxycarbonyl.

As discussed above, the surface tether comprises a cysteine residue or a suitable derivative thereof such as, for example, hydrazine. Preferably, the surface tether terminates in a cysteine residue, such that the receptor can be attached to the surface tether through the cysteine residue. For example, one end of the surface tether can be attached to a surface of the substrate, as discussed above, while the other end of the surface tether is terminated with a cysteine residue. The cysteine residue can serve as the point of attachment for the receptor, for example by reacting the C- or N-terminus of the cysteine residue with a receptor.

The cysteine residue can either be reacted with the spacer group prior to or after attaching the spacer group to the surface of the substrate. In a preferred aspect, the spacer group of the surface tether is first reacted with the surface of the substrate, and the cysteine residue is subsequently reacted with a reactive group present on the surface tether. A variety of suitable reactive groups can be present on the spacer, such as a nucleophile. In one aspect, the spacer comprises an amine as a reactive group that can couple to an activated cysteine residue. After reacting the cysteine residue with the spacer group to provide the surface tether, the cysteine group can then be reacted with a suitable receptor.

Mixtures of tethers for single molecule can create single molecule reaction sites with a doping of 1×10⁻⁴ to 1×10⁻⁶ of the FMOC protected tether into inert tether. Use of similar length and composition tethers also facilitates control site density. Backfilling of unreacted amine sites is typically with a small amine-reactive molecule. Typically succinimidyl tartrate is used but any other small mass (<300 mw) amine-reactive succinimide ester (NHS-ester) can be used. The resulting surface is a self-assembled monolayer with optically separated protected nucleopiles.

Deprotection of the FMOC leaves the nucleophile available to react to the addition of third tethering molecule. For example, a 2000 mw FMOC-amine-PEG-NHS, can be used, but other amine reactive tethers can also be used. Deprotection of the third tether reveals a nucelophile, typically an amine. A single round of peptide synthesis is used to couple a cysteine residue to the tether's amine. The surface is now immobilized cysteine at optically separated loci.

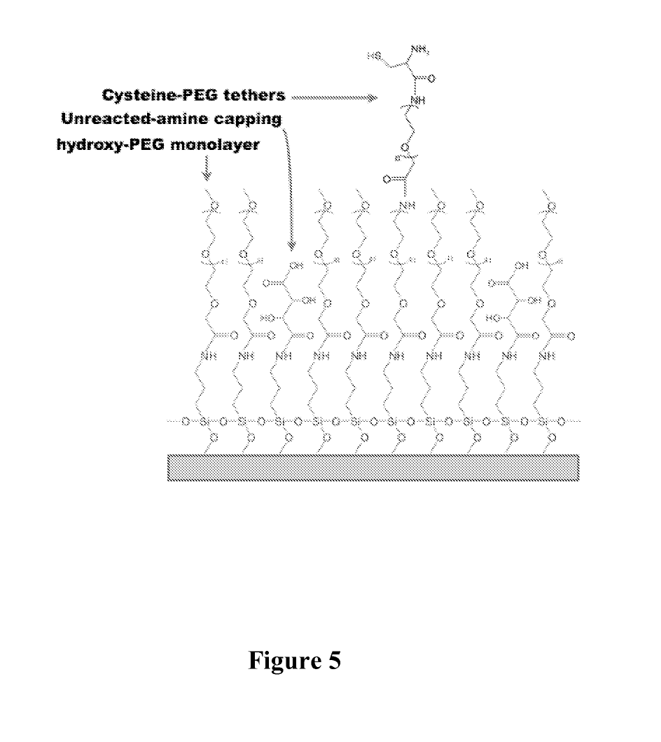

Thus, for example, an amidated glass surfaces can be reacted with a mixture of PEG-NHS oligomers. The PEG oligomer mixture is FMOC-protected amine PEG-NHS at less than 1×10⁻⁴ of the concentration of 16 nM PEG-NHS. Backfilling with the amine reactive molecule of succinimidyl L-tartrate blocks unreacted glass surface amines towards further reactions. Deprotection removal of the FMOC is performed and a second round of PEG-NHS reactions are performed. These additional PEGs position cysteines on long tethers extending into solution above the lower PEG monolayer. These cysteines are then used to capture and immobilize biomolecules at optically separated loci (FIG. 5).

In one aspect, the receptor that is immobilized to the surface through the surface tether can be represented by the following general formula:

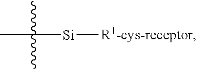

wherein R¹ is substituted or unsubstituted C₃-C₃₀, a polyethylene glycol polymer, polydextran, or a peptide linker. In a further aspect, the immobilized receptor and surface tether can be represented by the formula:

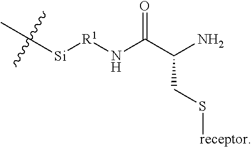

The receptor can be a variety of species that can interact with a desired analyte or ligand of interest. Examples include peptides, polypeptides, proteins, RNA, DNA, or carbohydrate. In one aspect, the receptor comprises a peptide, polypeptide, or protein azide. In yet another aspect, the receptor comprises a peptide, polypeptide, or protein thioester (or phosphenolthioester). In this example, a desirable peptide can be modified with an intein sequence that yields a terminal thioester group (or phosphenolthioester) on the peptide. The thioester can be reacted with the cysteine residue to attach the peptide to the surface tether.

The surface of the substrate can also comprise non-reactive residues that function to both uniformly space the surface tethers and to repel the analyte that is free in solution when carrying out single-molecule kinetic imaging. Examples of such non-reactive residues include without limitation alkyl cyanides, such as a cyano silane for use with a glass surface. The non-reactive residues do not comprise a cysteine residue and preferably react little, if any, with the cysteine residue. For example a non-reactive residue can be an alkyl cyano silane. The silane can anchor to the surface of the substrate, while the cyano group is distanced from the surface by the alkyl group.

Surface distribution of spacer groups (the surface tether precursor) can be controlled by varying the ratios of other non-reactive spacers to the surface tether precursor in a one pot reaction. For example, a suitable molar ratio of the non-reactive residue and the spacer group can be formulated to provide positioning of the cysteine tethers at either uniform monolayer distribution, or at optically-separated loci, or various surface densities in between. Thus, in some aspects, the receptor is immobilized on the surface in a particular pattern or in particular locations while in other aspects it is randomly immobilized.

Passivation of the non-reactive residue regions (i.e. alkyl cyano silane coated reagions) can decrease nonspecific adsoption interactions to the non-reactive residue regions. Examples of surface passivation agents are gelatin or serum albumin (i.e. BSA) or acylated derivatives thereof. These substances renders the non-reactive residue region substantially unreactive to ligand or analyte that is free in solution.

As discussed above, the surface tether can be bonded covalently to a receptor through the cysteine residue of the surface tether, such that the receptor is adequately suspended from the substrate surface and is accessible to a ligand or analyte that is free in a solution above the passivated substrate surface.

Methods for Making the Substrates

In one aspect, cysteine-derivatized surfaces are used to capture cysteine-reactive biomolecules. The surfaces are exposed to cysteine-reactive biomolecules. Nucleophilic attack of the cysteine's thiol creates a covalent bond cross-link to the biomolecule. The covalent bond immobilizes the biomolecule to the glass surface.

Prior to derivatizing the substrate surface, the substrate can be cleaned. Suitable cleaning methods include ozone cleaning, chemical etching, and the like. Once the substrate has been cleaned, the surface tether can be attached to the substrate surface. Generally, the surface tether can be attached to the surface by reaction of a surface group with a reactive group on the surface tether. For example, a silane on the surface tether can be reacted with surface hydroxyl groups on a glass slide.

The cysteine residue can be attached to spacer portion of the surface tether using standard peptide coupling techniques. In one aspect, the spacer group comprises a suitable nucleophile that can react with an activated cysteine residue to form an ester or amide bond with the cysteine residue. A variety of peptide coupling reagents can be used to attach the cysteine residue to the spacer group to provide the cysteine tether. Examples include phosphonium reagents such as BOP, PyBOP, BroP, PyBroP, and uronium reagents, such as HBTU, TBTU, TPTU, TSTU, TNTU, TOTU, HATU, HAPyU, TAPipU, BOI or acid chlorides or acid fluorides. The cysteine residue can be protected at the amino terminus prior to attaching the cysteine residue to the spacer portion of the surface tether. A variety of a amino protecting groups can be used, such as triphenylmethyl (trityl).

Oriented receptor immobilization (including uniform oriented receptor immobilization) is important for accurate binding affinities and rate determinations in affinity assays. Without oriented immobilization, binding heterogeneities occur that report non-native interaction strengths (a common problem with Biacore, which uses random immobilization of receptor). Thus disclosed herein are methods for immobilizing protein thioesters in uniform orientations on a substrate. It is understood and disclosed herein that by immobilizing the receptor to the substrate, very long kinetic trajectories involving multiple binding and unbinding events per molecule can be observed without the interference from photobleaching of the immobilized partner. Thus, the methods disclosed herein can be used to measure rare conformational states of the immobilized molecule. Moreover, the single molecule association and dissociation rates can be measured by the methods disclosed herein in addition to simple K_{d} measurement. This difference, though subtle, is significant because K_{d} is the measurement of the total rate of binding and unbinding whereas the present methods can further distinguish a fast binding followed by a slow unbinding from a slow binding and fast unbinding where the rate of the complete binding-unbinding cycle is the same.

As disclosed above, oriented immobilization can accomplished by crosslinking target molecules to tethered cysteines (see “Experimental Section, surface derivatization” for method details). Soluble peptide linkers are also suitable. The terminal amines are for attachment of a cysteine by using one round of solid-phase peptide synthesis (i.e. Cysteine-FMOC coupling). The resulting surface has well spaced cysteine molecules, which readily attack thioester bonds. When utilized, tethers terminating in cysteines can be used to capture protein thioesters. This capture results in a non-reversible peptide bond. Alternatively the cysteines can be used to captures thiol bearing biomolecules. These bonds are disulfide and are reversible by treatment small, soluble thiols such as DTT and BME. Alternatively the tether's amine can be reacted with a cross linking reagent such as a disuccinimyl-ester and allow capture of amine bearing biomolecules. Alternatively heterbifunctional cross-linkers can be used to irreversibly immobilize biomolecules containing thiols or carboxyl. The resulting biomolecules are immobilized at optically separated loci.

To create protein thioesters, the receptor protein sequences were fused to intein sequences. Inteins or protein introns are small protein fragments capable of self excision and fusing two or more peptides or proteins together. Herein, fusion constructs were expressed and purified via affinity chromatography. Induction of intein cleavage with mercaptoethylsufate resulted in elution of protein with a C-terminus thioester. Phosphothiophenol can also be used to induce intein cleavage and results in a protein phosphothioester. Reaction of the protein thioester (or phosphothioester) to the surface attached cysteine creates a covalently-bound, uniformly-oriented target molecule. The receptors can be immobilized to the surface tether using methods disclosed herein.

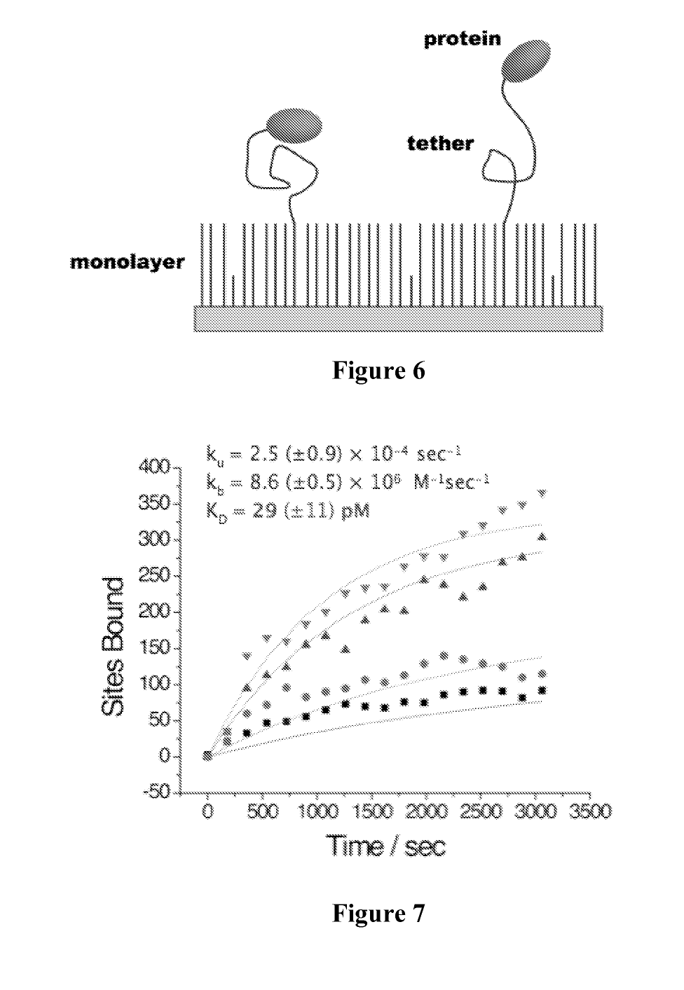

Thus, for example, a protein of interest is expressed as fusion construct to intein self-splicing domain. Purified fusion construct is cleaved from intein by reaction to sodium 2-mercaptoethanesulfonate (MESNA). MESNA reaction creates a protein thioester. The thioester reacts to surface-immobilized cysteine. A new thioester bond forms between the protein and the surface cysteine. The cysteine's amine then attacks the carbonyl of the thioester bond and an amine ester bond is formed. The resulting protein is immobilized to the glass surface by a stable, covalent peptide bond (FIG. 6)

As described herein, binding affinity interactions refers to the strength of binding as well as the rate at which a molecule will bind and release a target. It is understood that any art accepted method of making a measurement of the strength or rate of binding is suitable for the methods disclosed herein. For example, the binding affinity can be measured by determining the ratio of unbinding and binding rates. Alternatively, the Langmuir isotherm can be used to determine binding affinity. The Langmuir isotherm is an equation which relates the absorption of molecules on a solid surface to the concentration of a medium above the solid surface at a fixed temperature. It is also understood that the disclosed methods can be used to asses the rate of association and dissociation of the first molecule to the receptor.

In order to measure binding interactions, methods, such as fluorescence microscopy have been utilized in the art to visualize these interactions such that the measurements may be taken. The methods disclosed herein can use genetically encoded fluorophores or chemically attaching the fluorophores to the protein. Genetically encoded fluorophores have an advantage because the fluorophores are uniform in number and conformation with respect to the protein. In addition, the proteins are not subjected to chemical reactions that can affect their function. Binding heterogeneities can also occur with random labeling of the probe molecule. An example of a genetically encoded fluorphore is the use of a GFP fluorophore attached to the N or C-terminus of the probe ligand sequence which creates a uniformly labeled probe molecule. Tagging ligands with GFP has worked well for intermediate affinity interactions, but has become problematic for high affinity interactions (>nM) where the GFP bleaching rates approaches (or exceeds) the unbinding rates. To compensate for these undesirable photophysical characteristics of GFP, the disclosed pulsed-light excitation method allows the measurement of off rates that are slower than the bleaching rate of GFP.

Because pulse-light excitation extends the effective bleach rate but does not prevent bleaching events, the use of genetically encoded fluorophores can lead short measurement times and a loss of ability to visualize and measure many of the interactions. This leads to inaccurate results. Thus, it is contemplated herein that multiple fluorophores with more desirable photo-physical properties can be introduced at specific sites on ligand molecules. In one aspect, the multiple fluorphores comprise protein thioesters reacted with poly-fluorophore compounds designed with specific reactivity to thioester bonds. For example, the C-terminal intein (Ssp) was used to generate a C(GK)n peptide motif that was decorated with alexa-488 succinimide ester while bulk protein is bound to beads. Intein mediated peptide bond cleavage was then induced and resulted in elution of a thioester-reactive, N-terminal cysteine peptide containing multiple alexa-488 conjugates. In another aspect ligand molecules can be encoded with multiple sequence motifs conferring specific affinity to fluorogenic compounds. For example, FLASH reagents exist that allow site-specific incorporation of fluorophores with ideal photo-physical properties for KISMI of high affinity interactions. In yet another aspect, multiple fluorophore labeling of the zz-domain of protein A, followed by a clean-up on an antibody column to exclude molecules where labeling interferes with binding, can be used to provide a generic multifluor label for the constant region of an antibody, leaving the region of antibody recognition unaffected. Thus disclosed herein are methods wherein the molecule comprises a poly-fluorophore.

Nevertheless, traditional methods of fluorescence labeling and labels can be used in the methods disclosed herein. As used herein, a label can include a fluorescent dye, a member of a binding pair, such as biotin/streptavidin, a metal (e.g., gold), or an epitope tag that can specifically interact with a molecule that can be detected, such as by producing a colored substrate or fluorescence. Substances suitable for detectably labeling proteins include fluorescent dyes (also known herein as fluorochromes and fluorophores) and enzymes that react with colorometric substrates (e.g., horseradish peroxidase). The use of fluorescent dyes is generally preferred in the practice of the invention as they can be detected at very low amounts. Furthermore, in the case where multiple molecules are reacted with a receptor (in the case of a competition assay or in an assay to measure combination treatments), each molecule can be labeled with a distinct fluorescent compound for simultaneous detection. Labeled spots on the array are detected using a fluorimeter, the presence of a signal indicating an receptor bound to a specific molecule.

Fluorophores are compounds or molecules that luminesce. Typically fluorophores absorb electromagnetic energy at one wavelength and emit electromagnetic energy at a second wavelength. Representative fluorophores include, but are not limited to, 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein; 5-Carboxytetramethylrhodamine (5-TAMRA); 5-Hydroxy Tryptamine (5-HAT); 5-ROX (carboxy-X-rhodamine); 6-Carboxyrhodamine 6G; 6-CR 6G; 6-JOE; 7-Amino-4-methylcoumarin; 7-Aminoactinomycin D (7-AAD); 7-Hydroxy-4-I methylcoumarin; 9-Amino-6-chloro-2-methoxyacridine (ACMA); ABQ; Acid Fuchsin; Acridine Orange; Acridine Red; Acridine Yellow; Acriflavin; Acriflavin Feulgen SITSA; Aequorin (Photoprotein); AFPs—AutoFluorescent Protein—(Quantum Biotechnologies) see sgGFP, sgBFP; Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; Aminomethylcoumarin (AMCA); AMCA-X; Aminoactinomycin D; Aminocoumarin; Anilin Blue; Anthrocyl stearate; APC-Cy7; APTRA-BTC; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); Blue Fluorescent Protein; BFP/GFP FRET; Bimane; Bisbenzemide; Bisbenzimide (Hoechst); bis-BTC; Blancophor FFG; Blancophor SV; BOBO™-1; BOBO™-3; Bodipy492/515; Bodipy493/503; Bodipy500/510; Bodipy; 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; BTC; BTC-5N; Calcein; Calcein Blue; Calcium Crimson-; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CCF2 (GeneBlazer); CFDA; CFP (Cyan Fluorescent Protein); CFP/YFP FRET; Chlorophyll; Chromomycin A; Chromomycin A; CL-NERF; CMFDA; Coelenterazine; Coelenterazine cp; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM I Methylcoumarin; CTC; CTC Formazan; Cy2™; Cy3.18; Cy3.5™; Cy3™; Cy5.18; Cy5.5™; Cy5™; Cy7™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3′DCFDA; DCFH (Dichlorodihydrofluorescein Diacetate); DDAO; DHR (Dihydrorhodamine 123); Di-4-ANEPPS; Di-8-ANEPPS (non-ratio); DiA (4-Di 16-ASP); Dichlorodihydrofluorescein Diacetate (DCFH); DiD-Lipophilic Tracer; DiD (DilC18(5)); DIDS; Dihydrorhodamine 123 (DHR); Dil (DilC18(3)); I Dinitrophenol; DiO (DiOC18(3)); DiR; DiR (DilC18(7)); DM-NERF (high pH); DNP; Dopamine; DsRed; DTAF; DY-630—NHS; DY-635—NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (111) chloride; EYFP; Fast Blue; FDA; Feulgen (Pararosaniline); FIF (Formaldehyd Induced Fluorescence); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; Fluor X; FM 1-43™; FM 4-46; Fura Red™ (high pH); Fura Red™/Fluo-3; Fura-2; Fura-2/BCECF; Genacryl Brilliant Red B; Genacryl Brilliant Yellow 10GF; Genacryl Pink 3G; Genacryl Yellow SGF; GeneBlazer; (CCF2); GFP(S65T); GFP red shifted (rsGFP); GFP wild type′ non-UV excitation (wtGFP); GFP wild type, UV excitation (wtGFP); GFPuv; Gloxalic Acid; Granular blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1 low calcium; Indodicarbocyanine (DiD); Indotricarbocyanine (DiR); Intrawhite Cf; JC-1; JO JO-1; JO-PRO-1; LaserPro; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-lndo-1; Magnesium Green; Magnesium Orange; Malachite Green; Marina Blue; I Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxedidole; Noradrenaline; Nuclear Fast Red; i Nuclear Yellow; Nylosan Brilliant lavin E8G; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1; POPO-3; PO-PRO-1; PO-I PRO-3; Primuline; Procion Yellow; Propidium lodid (P1); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine: Phalloidine; Rhodamine Red; Rhodamine WT; Rose Bengal; R-phycocyanine; R-phycoerythrin (PE); rsGFP; S65A; S65C; S65L; S65T; Sapphire GFP; SBFI; Serotonin; Sevron Brilliant Red 2B; Sevron Brilliant Red 4G; Sevron I Brilliant Red B; Sevron Orange; Sevron Yellow L; sgBFP™ (super glow BFP); sgGFP™ (super glow GFP); SITS (Primuline; Stilbene Isothiosulphonic Acid); SNAFL calcein; SNAFL-1; SNAFL-2; SNARF calcein; SNARF1; Sodium Green; SpectrumAqua; SpectrumGreen; SpectrumOrange; Spectrum Red; SPQ (6-methoxy-N-(3 sulfopropyl)quinolinium); Stilbene; Sulphorhodamine B and C; Sulphorhodamine Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TON; Thiolyte; Thiozole Orange; Tinopol CBS (Calcofluor White); TIER; TO-PRO-1; TO-PRO-3; TO-PRO-5; TOTO-1; TOTO-3; TriColor (PE-Cy₅); TRITC TetramethylRodaminelsoThioCyanate; True Blue; Tru Red; Ultralite; Uranine B; Uvitex SFC; wt GFP; WW 781; X-Rhodamine; XRITC; Xylene Orange; Y66F; Y66H; Y66W; Yellow GFP; YFP; YO-PRO-1; YO-PRO 3; YOYO-1; YOYO-3; Sybr Green; Thiazole orange (interchelating dyes); semiconductor nanoparticles such as quantum dots; or caged fluorophore (which can be activated with light or other electromagnetic energy source), or a combination thereof.

A modifier unit such as a radionuclide can be incorporated into or attached directly to any of the compounds described herein by halogenation. Examples of radionuclides useful in this embodiment include, but are not limited to, tritium, iodine-125, iodine-131, iodine-123, iodine-124, astatine-210, carbon-11, carbon-14, nitrogen-13, fluorine-18. In another aspect, the radionuclide can be attached to a linking group or bound by a chelating group, which is then attached to the compound directly or by means of a linker. Examples of radionuclides useful in this aspect include, but are not limited to, Tc-99m, Re-186, Ga-68, Re-188, Y-90, Sm-153, Bi-212, Cu-67, Cu-64, and Cu-62. Radiolabeling techniques such as these are routinely used in the radiopharmaceutical industry.

Labeling can be either direct or indirect. In direct labeling, the molecule of interests includes a detectable label. In indirect labeling, an additional molecule or moiety is brought into contact with, or generated at the site of, the molecule. For example, a signal-generating molecule or moiety such as an enzyme can be attached to or associated with the detecting molecule. The signal-generating molecule can then generate a detectable signal at the site of the molecule. For example, an enzyme, when supplied with suitable substrate, can produce a visible or detectable product at the site of the molecule.

As another example of indirect labeling, an additional molecule (which can be referred to as a binding agent) that can bind to either the molecule of interest or to an antibody (primary antibody) to the molecule of interest, such as a second antibody to the primary antibody. The additional molecule can have a label or signal-generating molecule or moiety. The additional molecule can be an antibody, which can thus be termed a secondary antibody. Binding of a secondary antibody to the primary antibody can form a so-called sandwich with the first (or primary) antibody and the molecule of interest. The immune complexes can be contacted with the labeled, secondary antibody under conditions effective and for a period of time sufficient to allow the formation of secondary immune complexes. The secondary immune complexes can then be generally washed to remove any non-specifically bound labeled secondary antibodies, and the remaining label in the secondary immune complexes can then be detected. The additional molecule can also be or include one of a pair of molecules or moieties that can bind to each other, such as the biotin/avadin pair. In this mode, the detecting antibody or detecting molecule should include the other member of the pair.

Other modes of indirect labeling include the detection of primary immune complexes by a two step approach. For example, a molecule (which can be referred to as a first binding agent), such as an antibody, that has binding affinity for the molecule of interest or corresponding antibody can be used to form secondary immune complexes, as described above. After washing, the secondary immune complexes can be contacted with another molecule (which can be referred to as a second binding agent) that has binding affinity for the first binding agent, again under conditions effective and for a period of time sufficient to allow the formation of immune complexes (thus forming tertiary immune complexes). The second binding agent can be linked to a detectable label or signal-generating molecule or moiety, allowing detection of the tertiary immune complexes thus formed. This system can provide for signal amplification.

The methods disclosed herein are particularly well suited to determine the affinity for multi-subunit receptor complexes. Thus, disclosed herein, in one aspect, are methods wherein the receptor comprises a multi-subunit complex.

A known problem to the previously existing methods of determining binding interactions is that those methods were either unable to examine or provided inaccurate results when the binding affinity exceeded 1 nM. In particular, the methods often required that the molecule be diluted to allow measurements to take place; however, in diluting the molecule, uneven distribution of ligand molecules at the receptor interface led to inaccurate results. The methods disclosed herein are particularly suited for high affinity interactions. Thus, disclosed herein are methods wherein the binding affinity is greater than 10 nM. Also disclosed are methods wherein the binding affinity is greater than 1 nM. Also disclosed are methods wherein the binding affinity is greater than 100 μM. Also disclosed are methods wherein the binding affinity is greater than 10 pM. Also disclosed are methods wherein the binding affinity is greater than 1 pM. Also disclosed are methods wherein the binding affinity is greater than 100fM. Also disclosed are methods wherein the binding affinity is greater than 10fM. Also disclosed are methods wherein the binding affinity is greater than 1 fM. Thus for example, disclosed herein are methods wherein the binding affinity is between 1 nM and 100 pM. It is further understood that while the present methods are particularly adept at measuring affinity interactions greater than 1 nM, the present methods are also sufficient to measure binding affinities less than 1 nM, for example, 100 nM or 1 μM.

Excitation and visualization of fluorophores can be accomplished by any means known in the art, such as, for example, total internal reflection fluorescence (TIRF) microscopy. In TIRF, an evanescent wave is used to selectively illuminate and excite fluorophores in a restricted region of the specimen immediately adjacent to the substrate-water interface. TIRF uses pulsed light excitation and because electromagnetic field from the evanescent wave decays exponentially from the interface, penetration only occurs to a depth of only approximately 100 nm into the sample medium. Thus the TIRF enables a selective visualization of surface regions such as the basal plasma membrane (which are about 7.5 nm thick) of cells. Thus, disclosed herein are methods of measuring binding interactions using a pulsed light excitation strategy, such as, for example TIRF.

Alternative excitation and visualization of fluorophores can be accomplished by means of Biplane optics microscopy. Biplane optics direct wide-field images to a beam splitter to create two beam paths. The focus of one path occurs at the plane of receptor surface interface. The focus of the second path occurs at a plane higher than the receptor surface interface. The light from each path is directed to two separate regions on the chip of a CCD camera. The simultaneous images contain out of phase background light. Subtraction of the two images removes background light and only the differences in light remain. Since the Receptor surface light only creates a difference when a fluorescent ligand molecule binds a receptor, light signals of ligand binding are observed in a dark background (out of phase background canceled out).

The light source for excitation of the fluorophore can be any source capable of emitting light in the excitation range of the fluorophore or fluorophores in use. For example, if green fluorescence protein or other FITC derivative is used as the fluorophore, then the excitation source would need to operate at 488 nm. It is understood and contemplated herein, that the light source can produce a emission with a wavelength at 360 nm (near ultraviolet), 408 nm (such as a Krypton laser), 488 nm (such as an argon laser), 595 nm, or 633 nm (such as a HeNe laser). Other wavelengths such as far ultraviolet (less than 300 nm) and infrared (greater than 700 nm) can be used if the excitation of the fluorophore occurs at that wavelength. In addition to laser and diode excitation light sources, band passed filtered light from incandescent, halogen, metal-vapor, and fluorescent light sources could be used.

Detection of the light emission of the fluorophore can occur by any means known in the art. For example, a CCD camera can be used to record video images of binding and unbinding events.

### EXAMPLES

### Reagents and Materials

Toluene, methanol, N,N-dimethylformamide (DMF), and dichloromethane (DCM) spectrophotometric-grade solvents were obtained from Fisher Scientific (Hampton, N.H.). Toluene was dried over sodium for 24 h and filtered through a Millipore PTFE 0.2 μm filter (VWR, West Chester, Pa.) prior to use; methanol and DMF were used as received. Water was quartz-distilled and then filtered using a Barnstead NANOpure II system (Boston, Mass.) and had a minimum resistivity of 18.0 MΩ·cm. Phosphate buffered saline solution (PBS) was prepared using sodium phosphate dibasic (Mallinckrodt, Paris, Ky.) at 20 mM, where the pH was adjusted to 7.5 using 1.0 M sodium hydroxide and an ionic strength of 100 mM, adjusted with NaCl. Carbonate buffer was prepared using sodium bicarbonate (Mallinckrodt, Paris, Ky.) at a concentration of 20 mM, pH of 8.3 and an ionic strength of 100 mM, adjusted with NaCl. Sodium 2-mercaptoethanesulfonate (MESNA), N,N-Diisopropylethylamine (DIEA), piperidine, trifluoroacetic acid (TFA), and bovine serum albumin (BSA) were purchased from Sigma Aldrich and were used as received. Silane PEG5000 amine was purchased from Nanocs (New York, N.Y.). 2-Cyanoethyltriethoxysilane (CETES) was acquired from Gelest (Morrisville, Pa.). N-α-Fmoc-5-trityl-L-cysteine (Fmoc-trt-Cys), and Benzotriazole-1yl-oxy-tris-pyrrolidinophosphonium hexafluorophosphate (PyBOP) were purchased from Novabiochem (EMD Chemicals, Gibbstown, N.J.). Glass cover slips (Number 1, 22×22 mm) were purchased from Fisher Scientific (Hampton, N.H.).

### Example 1

### Preparation of Cysteine Derivatized Substrate

Glass cover slips were prepared for derivatization by first soaking in methanol for 30 minutes, allowing them to dry, and then placing them in a UV-ozone cleaning (Jelight Co. model 342) for 25 minutes on each side. Cleanliness of the slides was determined by checking with the use of water contact-angle measurement; a contact angle of <5° indicated that slides were sufficiently clean to produce uniform silane monolayers with minimal fluorescence background.

Modification of the slides was done out of refluxing toluene in a 500 mL three-armed round bottomed boiling flask with an attached condenser. With the addition of a cover slip holder (Thomas Scientific, Swedesboro, N.J.) it was possible for 10 cover slips to be derivatized at the same time. The reaction mixture consisted of 2-15 mM CETES and 1.0 pM to 1.0 nM silane PEG₅₀₀₀ amine in DMF or DMF:toluene solution. The reaction solution was kept at reflux for 24 hours with a continuous flow of nitrogen through the reaction vessel. Once the required time had elapsed the slides were removed from the reaction chamber rinsed with DMF and then baked in 120° C. oven for a minimum of 3 hours or up to 12 hours.

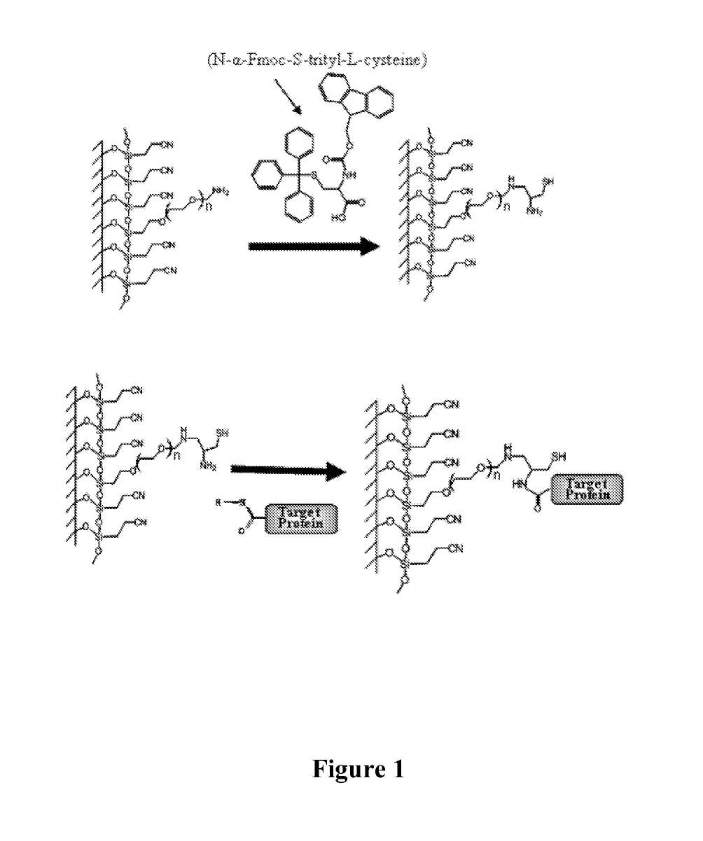

Immobilization of cysteine was accomplished according to FIG. 1. Immobilization of cysteine was accomplished by removing the slides from the oven, allowing them to cool in a desiccator, and adding 150 mL of DMF. To the DMF was added 0.002 moles of PyBOP, 0.001 moles of Fmoc-trt-cys, and 0.002 moles of (m,n-diisopropylethylamine (DIEA) were added. The reaction mixture (PyBOP, Fmoc-trt-cys, and DIEA) was allowed to mix for 5 min in 10 mL of DMF prior to addition to the slides. The solution was stirred for 1 hour after which the slides were rinsed 3 times for 10 minutes in fresh DMF. Deprotection of the cysteine was started by immersing the slides in a 20% piperidine/DMF solution for 30 minutes to remove the Fmoc protecting group, followed by rinsing 3 times for 10 minutes in fresh DMF. The slides were then immersed into DCM and rinsed twice for 5 minutes to remove any residual DMF before the removal of the trytl protecting group. After the DCM rinses the slides were immersed into a 5% TFA/DCM mixture for 15 minutes to remove the trytl group. The slides were then rinsed twice in DCM for 10 minutes followed by 2 rinses in methanol for 10 minutes. The final two rinses were in PBS buffer for 10 minutes.

### Example 2

### Determining Binding Site Density

Binding site density was determined by labeling amine-PEG silane with tetramethylrhodamine succinimidyl ester (TMR-SE). Three coverslips were taken form each batch of modified slides before cysteine immobilization for labeling. The TMR-SE labeling reaction with amine reactive sites was accomplished through the use of succinimidyl ester chemistry (Wayment, J. R.; Harris, J. M. Analytical Chemistry 2009, 81, 336-342, Houlne, M. P.; Sjostrom, C. M.; Uibel, R. H.; Kleimeyer, J. A.; Harris, J. M. Analytical Chemistry 2002, 74, 4311-4319; Charles, P. T.; Conrad, D. W.; Jacobs, M. S.; Bart, J. C.; Kusterbeck, A. W. Bioconjugate Chemistry 1995, 6, 691-694) where the labeling reagent reacts with surface amines to form a peptide bond to the surface immobilized amine. A stock solution of 3-mg TMR-SE in 3-mL DMF was prepared and kept desiccated at −20° C. until use. An aliquot of the TMR-SE stock solution was diluted 1:125 in DMF (7.5 μM), and the amine-modified surfaces were reacted in this solution for 1 hour and then rinsed twice in DMF and four times in methanol for 20 min each. The derivatized cover slips were stored in methanol in the dark prior to their examination by TIRF microscopy.

Reactive amine functional groups on PEG tethers were immobilized at low surface densities on glass by self-assembly of mixed silane monolayers from solutions containing very low concentrations of amine-PEG₅₀₀₀-triethoxysilane and a much higher concentration of 2-cyanoethyltriethoxysilane (CETES). The modified coverslips were rinsed in toluene and methanol to eliminate excess silane reagent, following which they were heated to 120° C. for a minimum of 3 hours to promote condensation reactions with the surface and cross-linking of the monolayer film. The concentrations of amine-PEG₅₀₀₀-silane (1.2 pM) to 2-cyanoethyltriethoxysilane (15 mM) corresponded to a concentration ratio of 8×10⁻¹¹. If the amine-binding site concentrations in the monolayer corresponded directly to these dilution factors, then one would expect the amine site density to be 0.057 μM⁻² based on the molecular density of self-assembled and cross-linked alkylsiloxane monolayers of ˜0.23 (±0.02) nm²/silane determined by infrared absorption and X-ray reflection measurements.

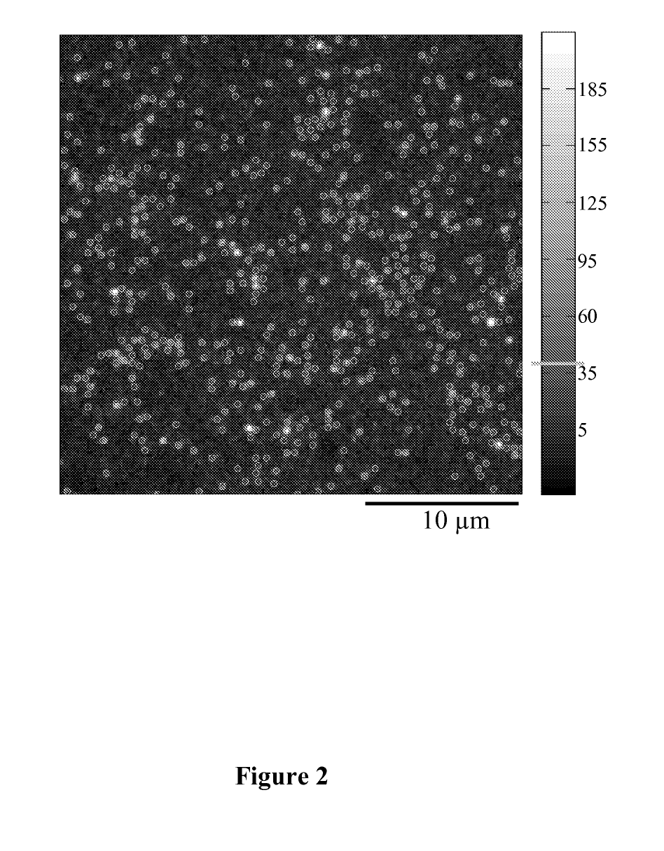

To measure the surface density of amine-PEG₅₀₀₀-silane molecules immobilized in the cyanoethylsilane monolayer, the amine groups were reacted with tetramethylrhodamine succinimidyl ester (TMR-SE) in DMF for 60 min. The reacted slides were rinsed in DMF and methanol, and then imaged using total internal fluorescence microscopy using 1.5 mW of laser power and a 250 ms integration. The threshold for counting single molecule spots was determined from the background intensity and noise level. The background level was μ_{B}˜14 photoelectrons, while the pixel-to-pixel variation in background counts had a standard deviation, σ_{B}=5.6 photoelectrons, equivalent to the fundamental shot-noise for the observed background level. The threshold for counting molecules was set at Lc=36 photoelectrons, which is 4-times σ_{B} above μ_{B} FIG. 2, making the probability of false positive counts arising from the variation in the dark background negligible, αₚᵢₓₑₗ<0.014, which is the probability of any given pixel being above background; this value seems small but the large number of pixels per frame (122,500) indicates that a large number of false positives would be observed in each frame (˜1,700). To reduce this number, the spot counting algorithm requires three adjacent pixels be above threshold to assure that the event derives from a spot having a size equivalent to the point spread function. The probability of random events producing three adjacent pixels that are all above Lc is α₃₋ₚᵢₓₑₗₛ<0.014³=2.7×10⁻⁶, which lowers the false rate to be α<0.33 per frame. This means that in every ˜3 frames, one count will be a false positive, these events are further filtered out by requiring that molecules remain for more than one frame. The detected TMR exhibited a distribution of intensities above the background with an average peak intensity μ_{P}=48 photoelectron counts and a standard deviation, σ_{P}=8 photoelectrons. The intensity threshold for counting molecules, Lc=36 photoelectrons, is ˜2 σp below the average peak intensity, leading to a false negative rate of β<0.025.

The surface concentration of amine-bound tetramethylrhodamine molecules was determined by counting the number of located TMR molecules above threshold in seven different 58 μm×58 μm areas. The binding site density determined using TMR immobilization to the primary amine was 0.44 (±0.03) molecules per μm². The binding site density is 8-times higher than expected based on the concentration ratio of the amine-PEG₅₀₀₀-silane relative to the cyanoethyl silane in the reaction solution used to create the self-assembled monolayer. This discrepancy may be due to the poorer solubility of the amine-PEG₅₀₀₀-silane in the toluene/DMF solution, which would preferentially lead to adsorption and binding of the amine-PEG₅₀₀₀-silane reagent.

### Example 3

### Protein Immobilization

Bead bound protein was incubated in 20 mM MESNA and PBS buffer for 1.5 hrs at room temperature immediately prior to slide incubation. After the allotted incubation time the beads were spun down in a centrifuge and the supernatant was collected. Before immobilization of the protein, the glass surface was passivated using a 0.1 mg/mL solution of BSA in PBS for 20 minutes. Slides were immersed in a carbonate buffer (pH 8.3) for coupling of the protein thioester to the surface immobilized cysteines. The collected supernatant was added to the modified slides and the mixture was stirred for 1.5 hours. Once the reaction was complete the slides were rinsed in PBS buffer twice for 10 minutes and then left overnight.

In order to use amine-PEG tethers as sites for the immobilization of a target protein, the terminal amine groups were cysteinylated using solid phase peptide procedures (Houlne, M. P.; Sjostrom, C. M.; Uibel, R. H.; Kleimeyer, J. A.; Harris, J. M. Anal Chem 2002, 74, 4311-4319). The terminal cysteine could further be reacted to the N-terminal thioester of the syntaxin protein to covalently immobilize it to the PEG tethers. A thioester will form a disulfide bond with the free thiol of cysteine; it then goes through an S—N acyl shift to form a stable peptide bond with free cysteine (Rusmini, F.; Zhong, Z.; Feijen, J. Biomacromolecules 2007, 8, 1775-1789).

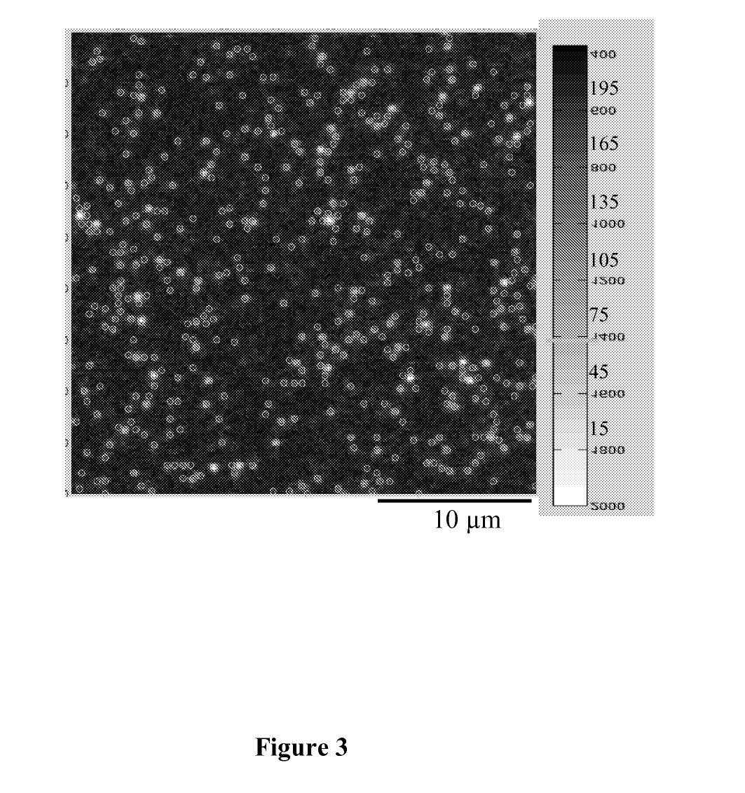

To determine the density of immobilized cysteine sites on the PEG tethers, green fluorescent protein (GFP)-labeled synaptobrevin was covalently attached to the cysteine groups by reaction with a thioester of the labeled protein. The modified coverslip was then assembled into the microscopy flow cell on the TIRF microscope and illuminated with 488 nm laser radiation, which matched the excitation maximum of the eGFP variant used. GFP modified slides were imaged using 12 mW of laser power and a 250 ms integration time. The threshold for counting single GFP molecules was determined from the noise level and intensity of the background. The background intensity was μ_{B}˜36 photoelectrons, while the pixel-to-pixel variation in background counts had a standard deviation, σ_{B}=7 photoelectrons. The threshold for counting molecules (Currie, L. A. Analytical Chemistry 1968, 40, 586-593) was set at Lc=59 photoelectrons, which is 3-times σ_{B} above μ_{B}, FIG. 3, leading to an expected false positive rate of α<0.0095 per pixel or α<0.1 per frame with the 3-adjacent-pixel requirement (see above). The GFP molecules exhibited a distribution of intensities that was best fit to a Gaussian distribution convoluted with a single-sided exponential (Dyson, N. A.; Smith, R. M. Chromatographic Integration Methods, 2nd ed.; Royal Society of Chemistry: London, 1998). The width of the distribution and the high-intensity tail is dominated by the exponential decay, were τ=6 photoelectrons, while the standard deviation of the Gaussian, σ_{G}=5 photoelectrons, dominates the histogram at low-intensities. The probability of false negative events, that is failing to detect a surface immobilized GFP-labeled synaptobrevin, was determined by comparing the area of the fitted distribution below the threshold with the total area under the curve; the resulting false negative probability is small, β˜3×10⁻³. The cysteine site density determined by immobilizing GFP-synaptobrevin was determined to be 0.41 (±0.02) molecules per μm². This density corresponds, within the uncertainty of the measurement, to the same density determined for the amine-PEG tethers; indicating that essentially all amine binding sites are converted to protein immobilization sites.

To determine whether non-specific interactions of the thioester protein with the BSA passivated surface, account for any of the observed GFP-synaptobrevin above, GFP-labeled synaptobrevin was allowed to interact with a BSA treated, non cysteine modified PEG₅₀₀₀-cyano surface. BSA treated slides were allowed to interact with the GFP-labeled protein for 1 hour after which they were rinsed using the same protocol used to rinse the excess thioester protein after immobilization to cysteine. The slides were then assembled into a flow cell and imaged on the TIRF microscope using the same conditions as above, 12 mW of laser power and a 250 ms integration time frames were acquired and examined for 3 slides and no fluorescent spots were observed above the 3 to 5 per frame that are observed in the blank modified glass slides. We therefore can assume that the GFP-labeled synaptobrevin seen on the cysteinylated coverslips was indeed immobilized to the surface through the cysteine tether and not adsorbed to the BSA surface.

### Example 4

### Preparation of Synaptobrevin and GFP-Syntaxin

Plasmid construction: Plasmids were constructed in pTWIN vector system. pCH21 (eGFP-MRM-inteinMxe-CBD) was generated by cloning a PCR product from the eGFP plasmid (pd2eGFP) with primers oCEH48/oCEH50 then cloned NdeI/SapI into pTWIN1 vector (NEB, inc). pVJ04 (SYX(1-265)-GFP-inteinMxe-CBD) is derived from PCR of syntaxin (SYX) (cDNA clone pNA21) with oCEH24/oCEH25 then cloning XbaI/Nde into pCH21. pCH38 (CBD-inteinSSP-SYX(1-265)-GFP-6His) was derived from PCR on pVJ04 with oCEH1 15/oCEH1 16 and cloned SapI/PstI into pTWIN1. pVJ16 (SNB(1-94)-inteinMth-CBD) was generated from PCR on synaptobrevin (SNB) (cDNA clone pMH410) with oCEH62/oCEH78 and clone NdeI/SapI into pTWIN2. All PCR products generated using Phusion polymerase (NEB, Inc) and plasmid sequences were confirmed via DNA sequencing.

TABLE 1
Oligonucleotide List

| | | Sequence |
|---|---|---|
| Name | Sequence | Identifier |
| CEH25 | ctttgtttagcagcctaggtattaatcaat | SEQ ID NO 1 |
| | tagtg | |
| CEH26 | atcccgcgaaattaatacgactcactatag | SEQ ID NO 2 |
| CEH48 | GGTGGTGGTCATATGGTGAGCAAGGGC | SEQ ID NO 3 |
| | GAGGAGCTGTT | |
| CEH50 | GGTGGTTGCTCTTCTGCACATACGCAT | SEQ ID NO 4 |
| | CTTGTACAGCTCGTCCATGCCG | |
| | AGAGTGA | |
| CEH62 | ggaggaggacatATGGACGCTCAAGGAGAT | SEQ ID NO 5 |
| | GCCGGCGCACAG | |
| CEH78 | ggtggttgctcttctgcacatacgcatTTT | SEQ ID NO 6 |
| | GATGTTCTTCCACCAATACTTGCGCTTCAG | |
| | GGT | |
| CEH1 15 | GGTGGTTGCTCTTCCAACATGACTAAG | SEQ ID NO 7 |
| | GACAGATTGTCCGCTTTAAAAG | |
| CEH1 16 | GTGGTCTGCAGTTAgtgatggtgatggtg | SEQ ID NO 8 |
| | atgCTTGTACAGCTCGTCCATGCCGA | |

### Example 5

### Protein Expression and Purification

Impact-TWIN expression system (NEB, Inc) allows native terminus purification of recombinant proteins via cleavage with DTT (C-terminal intein fusions; plasmids: pCH21, pVJ04 pVJ16) or acidic conditions (pH 6, N-terminal intein fusions; plasmid pCH38). Additionally, thiol-reactive constructs are generated by cleaving C-terminal intein fusions with MESNA to create a protein thioester. Proteins were expressed from all constructs with IPTG induction (1 mM, A600=˜500) for 12 hrs at 20° C. Harvested cell pellets were frozen to −80° C. until used. Cell pellets (eq. 250 ml culutre) were resuspended in 25 ml of 2× Cellytic Express (SigmaAldrich, Inc) in M9 media solution. After lysis at room temperature for 15 min, lysate was vortexed 1 min at max then transferred to ice and incubated for 15 min. Lysates were further vortexed to shear DNA until viscosity approached a minimum (˜1 min). Lysates were centrifuged in SS-34 rotor at 15K rpm for 20 min and supernatant was harvested. For C-terminal intein fusion, lysates were bound to 2 ml of 50% suspension of chitin beads (NEB, inc) by incubation on a nutator for 2 hrs at 4° C. beads were settled by low speed centrifugation (˜1000 rpm) and recovered into disposable columns (biorad, inc). Bead bed was washed with 10 column volumes (CV) of Salt Wash Buffer (50 mM Na-Hepes at pH 7.3, 50 mM NaCl, 1% Trition X100) then 10 CV of Resuspension Solution (10 mM Na-Phosphate pH 7, 2 mM NaAzide). Bead bound proteins were eluted as native carboxyl terminus proteins (pCH21 and pVJ04) via 12 hr incubation in DTT elute (100 mM Na-Phosphate at pH 8, 20 mM DTT). For the N-terminal intein fusion (pCH38), clarified cell lysate was first purified on 2 ml of 50% His Select beads (Sigma, inc.), eluted in Salt Wash Buffer at 200 mM imidazole (pH ˜8.5), then purified on 2 ml of 50% chitin beads with Salt Wash/Imidazole buffer. The protein was eluted by incubation in acidic conditions (50 mM Hepes at pH 6, 500 mM NaCl, 1 mM EDTA). All proteins eluted as native termini were exchanged (2×) into Resuspension Solution via ultrafiltration centrifugation (Biomax 15, 30,000 MWCO). To create SNB-thioester (pVJ16), the construct was expressed and purified as above for C-terminal intein fusion constructs.

### Example 6

### Single-Molecule Imaging

Imaging of the GFP labeled syntaxin binding to immobilized synaptobrevin protein was accomplished using an Olympus IX71 microscope operated in TIRF mode. The GFP label was excited and imaged through an Olympus plan apo 60×1.45 NA, oil-immersion TIRF objective with a 1.6 magnifier in place, making the apparent magnification of the objective 96×. Excitation of the sample was achieved using an argon ion laser (coherent, model Innova 300) operated at 488 nm and coupled into the microscope using a polarization maintaining single-mode optical fiber. Total internal reflection was achieved by translating the fiber horizontally, which in turn moved the position of the incoming laser beam (5 mW) to the edge of the objective until internal reflection was observed at the interface between the coverslip and the buffer solution. Emitted fluorescence was collected back through the same objective and passed through a dichroic beam splitter and band-pass emission filter (Chroma Z514RDC and HQ560/50m, respectively) and imaged using an Andor IXON camera. TIRF images were an integration time of 250 ms, images were either collected every second or continuously. Andor IQ software was used to collect images; the area of image acquisition was set at 58 μm×58 μm.

Kinetic experiments were performed by mounting a modified coverslip into a microscopy flow cell and illumination with 488 nm laser radiation on a TIRF microscope for 10 minutes; this was done to photobleach any residual fluorescent spots from the glass substrate prior to image acquisition. The synaptobrevin coverslip was then exposed to the relevant concentration (0.085-2.2 nM) for either 25 minutes in the case of intermittent imaging or 17 minutes when continuous imaging was performed. The binding, unbinding, and affinity constant for the syntaxin—synaptobrevin complex were determined from the residence times of single molecule events.

### Example 7

### Data Analysis

Images are first processed by locating single molecules in each video frame using a custom threshold based detection method. The detection criteria, which are governed by the parameters of the point-spread function, require that at least 3 adjacent pixels brighter than an intensity set at 49.5 photoelectrons or 4 times the standard deviation of the background. By requiring 3 adjacent pixels to be above the set threshold the influence of cosmic rays and other discrete, non-molecular events on the counting results are greatly reduced. Individual binding site locations are located by correlating single molecule coordinates within ±1 pixel (167 nm) precision across multiple video frames. The density of binding sites observed at high antibody concentrations agrees well with the density of fluorophores covalently bound to surface PEG-cysteine sites on equivalent slides using tetramethylrhodamine labeling. Binding traces, vectors indicating the binding state of each site for each frame of the video, are generated by correlating the binding site coordinates with the list of located molecule coordinates in each frame. In order to correct for aberrant photochemical behavior, such as photoblinking, the data is filtered to remove brief unbound states of a single video frame. From these binding traces the bound state lifetimes, and the fraction of sites bound can be determined. Unbound state lifetimes are measured by recording the time duration of every unbinding event. Histograms of the unbound state survival times are plotted and fit to an exponential decay function to determine their unbinding time constants.

Affinity measures have been made by the KISMI method for interactions strengths as weak as 20 nM (Z-domain affinity to rabbit polyclonal antibody) and as high as 1.7 μM (formation of a SNARE complex from binding of synaptobrevin to the acceptor complex of syntaxin linked to SNAP-25).

### Example 8

### Extension of KISMI Technology to Polyethylene Glycol (PEG) Surfaces and Tethers

Methods

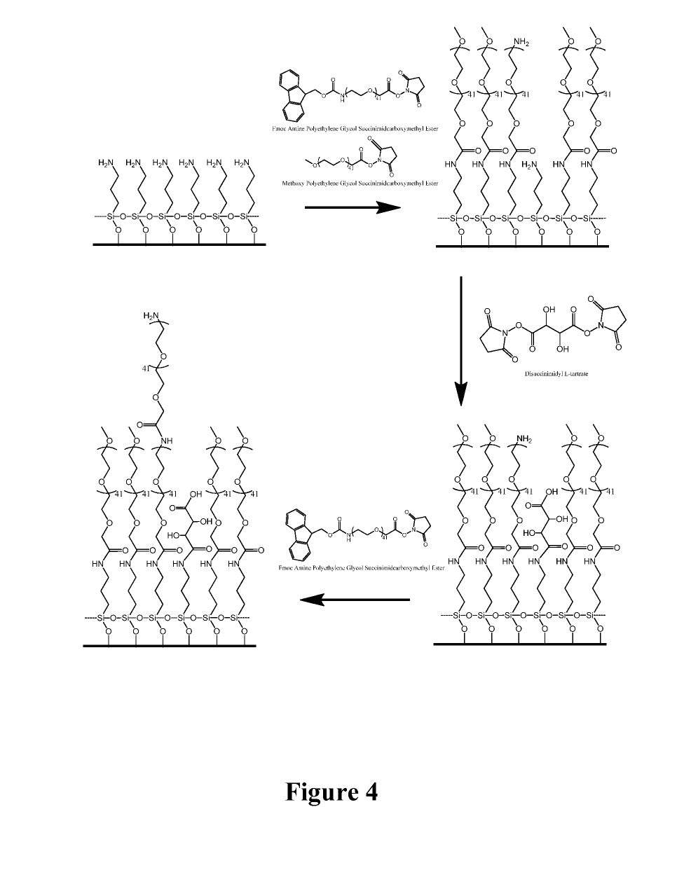

The grafting of polyethylene glycol molecules to a glass, silicon oxide, or quartz substrate can be accomplished with succinimidyl ester binding chemistry to an amine-silane monolayer (FIG. 4). Twelve 22 mm by 22 mm number 1.5 borosilicate glass cover slides were prepared for polyethylene glycol derivatization by first cleaning using the methods outlined by Kern and Puotinen. The cleaned slides were rinsed in methanol, vacuum dried for no less than one hour, and immediately amine functionalized (i.e., aminated). Cover slides were passivated in a solution of freshly prepared 1% 3-Aminopropyl triethoxysilane (APTES) in deionized water for 5 mins followed by 2 rinses in absolute ethanol. The APTES was covalently annealed and cross-linked to the glass substrate by incubating the cover slides at 150° C. for 12 hours.

Polyethylene glycol (PEG) chains were grafted onto the aminated surface by reacting the amine functionalized slides in 150 mL of a 16-nM PEG solution with mole ratios ranging from 1:1×10⁻⁴ to 1:1×10⁻⁶ of 2000 molecular weight methoxypolyethylene glycol succinimidyl carboxymethylester (m-PEG-NHS) and 9H-fluoren-9-yl methoxycarbonyl protected-amine polyethylene glycol succinimidyl carboxymethyl ester (Fmoc-N-PEG-NHS) in dichloromethane for 24 hours. Unreacted surface amines at the glass surface are then passivated with the addition of 3 nmol of disuccinimidyl L-tartrate into the reacting PEG solution for an additional 24 hours.

In order to better mimic solution-binding conditions for protein-protein binding sites, immobilized proteins are tethered on a 2000 molecular weight, ˜15 nm, PEG tether. This is achieved by first removing the Fmoc protecting group by reacting the slides in a solution of 5% piperidine in dichloromethane for 1 hour followed by 3 rinses in dichloromethane. Attaching a PEG tether to the free amine was achieved by reacting the slides in a 16 nM solution of 2000 molecular weight Fmoc-N-PEG-NHS in dichloromethane for 2 hours, and then rinsing 3 times in dichloromethane. The Fmoc deprotection of the tethered amine was accomplished by the reaction of the slides with a 5% solution of piperidine in dichloromethane for 1 hour followed by 3 rinses in dichloromethane (FIG. 4).

To provide a reactive group to bind target proteins, cysteine was immobilized to the PEG-tethered amines by adding 0.5 mmols of Benzotriazole-1yl-oxy-tris-pyrrolidiono phosphonium hexaphosphate (PyBOP), 0.2 mmols of Fmoc-5-trityl-Cysteine (Fmoc-trt-Cys), and 1 mmol Diisopropylethylamine (DIEA) to 5 mL of N,N-dimethylformamide and vigorously shaking for 5 minutes. Once the solution has turned a pale yellow, it was added to a beaker containing 150 mL of dichloromethane and the modified slides. After reacting for 1 hour the slides were rinsed 3 times in dichloromethane. The deprotection of cysteine was achieved by first the removal of the Fmoc protecting group by reacting the slides in a 5% piperidine in dichloromethane solution for 1 hour followed by 3 rinses in dichloromethane. Secondly, the removal of the trityl protecting group was performed by reacting the slides in a 1.5% trifluoroacetic acid (TFA) in dichloromethane solution for 15 minutes followed by 3 rinses in dichloromethane, 3 rinses in methanol, and finally 3 rinses in 20 mM, pH 7.5 phosphate buffered saline (PBS). Finally the slides were transferred into a 0.1 mg/mL solution of bovine serum albumin (BSA) in 20 mM, pH 7.5, PBS in preparation for protein immobilization.

Immobilization of protein to the surface bound cysteine was done through solid phase peptide synthesis. Syntaxin/synaptobreven expressed with reactive thioester on chitin beads was cleaved from the beads by incubation in 20 nM sodium 2-mercaptoethanesulfonate (MESNA) in 20 mM, pH 7.5 PBS for 1.5 hours. The cysteinilated slides were then transferred to carbonated buffer (pH 8.3), and the supernatant from the spun down beads was added to the solution. The slides were allowed to react for 2 hours, after which they were rinsed 2 times with 20 mM, pH 7.5 PBS and allowed sit over night. Approximately 12 hours after the immobilization of protein, the slides are then rinsed for 1 hour in 10 mM dithiothreitol (DTT), and twice in 20 mM, pH 7.5 PBS prior to use (FIG. 5).

Results

Reaction of APTES out of water was reported to produce maximum amine functionality on a glass surface; however, because this is an uncommon practice due to competition with silane hydrolysis. Prior to the disclosure herein, this practice warranted speculation to whether or not a covalently attached APTES monolayer can actually be established. The formation of an APTES monolayer was tested with the use of ellipsometry. Silicon wafers were subjected to the same reaction conditions as the glass cover slips and then measured for film thicknesses. The reported thickness for a monolayer of APTES is 7 Å, silicon wafers treated with a water/APTES solution ranged from 7.0 to 9.3 Å in thickness. Because the overall objective was to create a surface resistant to non-specific interactions, the confirmation of accessible amine functionality was qualitatively assessed by the ability to successfully graft PEG to the surface, this was tested by the final surface's ability to minimize non-specific interactions.

Protein repellency of the PEG modified slides was quantified with methoxy capped tethers in substitution of amine capped tethers. Because the interest is to measure the affinity between anti-syntaxin/syntaxin, and titrations for affinity measurements are typically carried out at concentrations surrounding the K_{d}, the quantification of non-specific interactions was measured at concentrations triple the K_{d}. The slides were tested against anti-syntaxin-labeled with oyster 550 for non-specific adsorption to the surface. It was determined that a non-specific interaction of <5% was sufficient to accurately measuring biomolecular affinities. Objective based total internal reflection fluorescence using a 514 nm argon ion laser radiation as the excitation source was utilized to measured surface interactions. Non-specific interactions were measured by first bleaching a PEG slide, within a flow cell containing 20 mM, pH7.5 PBS, in order to reduce background signal for 15 minutes. Afterwards, a 150 μM solution of anti-syntaxin-oyster 550 was injected into the flow cell. The sample was imaged with 1.5 mW of 514 nm laser radiation at 250 msec integrations every 5 minutes for 1 hour. Non-specific interactions were analyzed using a counting program. The analysis for non-specific adsorption resulted 3 to 9 events per 2.84 mm² frame. This result indicates that approximately 500 to 1000 binding sites per slide was adequate for kinetic measurements; the number of non-specific interaction are below 5% of the total possible observed events, which is below the limit of detection for the counting program

The ability to control site density relies on the ratioed PEG molecules being similar in size. Unequal size PEG molecules exhibit different rates of reactive succinimidyl ester groups finding and reacting to surface amines. Rationing of 2000 molecular weight mPEG-NHS with 3400 molecular weight Fmoc-N-PEG-NHS resulted in uncontrollable site density; however, when the ratio of PEG molecules were of the same molecular weight, site density became controllable. The determination of site density was conducted using synaptobrevin labeled with green fluorescent protein-(GFP) expressed as a reactive thioester on chitin beads. The synaptobrevin was cleaved from the chitin beads by incubation in 20 mM MESNA in 20 mM, pH 7.5 PBS for 1.5 hours. A deprotected cysteine immobilized slide was photo bleached with 488 nm argon ion laser radiation in a flow cell containing 20 mM, pH 8.3 citrate buffer for 15 minutes. After photo bleaching was concluded, the laser source was blocked and 50 μL of the supernatant collected from the cleaved synaptobrevin-GFP was injected into the flow cell. The resulting solution was allowed to react for 1.5 hours after which 20 mM, pH 7.5 PBS was flowed at a rate of 2 mL/hour through the cell for 1.5 hours. Under continuous flow, the slide was imaged using 1.5 mW of 488 nm argon ion laser radiation as the excitation source at 250 msec integration every 5 minutes for 2 hours. The video data were then analyzed, using a counting program, to find a plateau where the number of spots remained constant for 1 hour; this was considered to be the number of immobilized sites available on the surface. It was determined that the ratio of Fmoc-N-PEG-NHS to mPEG-NHS at 1:1×10⁵ would provide ample sites to perform kinetic measurements.

Protein-binding kinetics measurements were carried out using syntaxin immobilized slides against anti-syntaxin labeled with oyster 550 in solution using and excitation of 514 nm laser light. A slide was loaded into a flow cell containing 1 nM BSA and 10 mM DTT in nitrogen purged 20 mM, pH 7.5 PBS buffer. The slide was then photobleached with 13 mW of 514-nm laser radiation for 15 minutes. Concentrations of anti-syntaxin oyster 550 ranging from 10 to 80 μM were injected and then imaged with 1.5-mW 514-nm laser radiation at 250-msec integrations every 3 minutes for 2 hours. The resulting image stacks were analyzed using the counting program previously described. The accumulation results were fit to equation (1), simultaneously fitting all curves by varying k_{bind} and k_{unbind} (FIG. 7)

$$θ ( t ) = ( \frac{{k}_{bind} C}{{k}_{bind} C + {k}_{unbind}} ) [ 1 - exp ( - ( {k}_{bind} C + {k}_{unbind} ) t ) ] \qquad ( 1 )$$

where θ(t) is the fraction of bound sites versus time, θ(t)=Γ(t)/Γₒ where F(t) is the number of occupied sites versus time and Fₒ is the available site density, which was found to be 585 by prior counting of site densities, and C is the concentration of anti-syntaxin oyster 550-labeled in each experiment. The binding rate (on rate), k_{bind}, was determined to be 8.6 (±0.5)×10⁶ sec⁻¹ M⁻¹, and the unbinding rate (off rate), k_{unbind}, 2.5 (±0.9)ₓ10⁻⁴ sec⁻¹ M⁻¹.

$${K}_{d} = \frac{{k}_{unbind}}{{k}_{bind}} \qquad ( 2 )$$

Using equation (2) it was determined that the anti-syntaxin/syntaxin interaction, K_{d} of 29 (±11) pM.

What is claimed is:

1. A method of determining the kinetic binding properties of a first molecule comprising contacting one or more receptors with the first molecule and measuring the binding affinity interaction of the molecule, wherein the one or more receptors are covalently bound to the surface of a substrate.

2. The method of claim 1, wherein the concentration of the receptor on the surface of the substrate is the same as or less than the concentration in bulk solution

3. The method of claim 1, wherein the affinity is measured by determining the ratio of unbinding and binding rates.

4. The method of claim 1, wherein the affinity is measured by a Langmuir isotherm.

5. The method of claim 1, wherein the binding and unbinding rates are measured from video images of the binding and unbinding events.

6. The method of claim 1, wherein the binding and unbinding events are detected by monitoring fluorescence.

7. The method of claim 6, wherein the fluorescence is excited by a pulsed-light excitation strategy.

8. The method of claim 1, wherein the first molecule is a genetically-encoded fluorophore fusion construct.

9. The method of claim 1, wherein the first molecule is a poly-fluorophore.

10. The method of claim 1, wherein the first molecule comprises a green fluorescence protein fluorophore attached to the N or C-terminus of a probe.

11. The method of claim 1, wherein more than one fluorophore is used.

12. The method of claim 1, wherein the receptors are immobilized on the surface of the substrate in an oriented manner.

13. The method of claim 12, wherein the orientation is uniform.

14. The method of claim 12, wherein the receptors are immobilized by cross-linking the target molecules to tethered cysteines.

15. The method of claim 1, wherein the binding affinity interaction is at least 1 nM.

16. The method of claim 15, wherein the binding affinity interaction has an affinity between 1 nM and 100 pM.

17. The method of claim 15, wherein the binding affinity interaction has an affinity greater than 100 pM.

18. The method of claim 1, wherein the binding affinity interaction is less than 1 nM.

19. The method of claim 1, wherein the first molecule is a protein, DNA, RNA, or carbohydrate.

20. A substrate comprising one or more receptor tethered to a surface thereof through a surface tether; wherein the receptor is covalently bonded to the surface tether through a cysteine residue; and wherein the surface tether is covalently bonded to the surface of the substrate.

21. The substrate of claim 20, wherein the substrate is glass, quartz, silicon dioxide wafers, or a gold-coated surface.

22. The substrate of claim 20, wherein the cysteine residue is not directly covalently bonded to the surface of the substrate.

23. The substrate of claim 20, wherein the receptor is a polypeptide or peptide.

24. The substrate of claim 20, wherein the receptor is a polypeptide or peptide thioester.

25. The substrate of claim 20, wherein the substrate further comprises a surface passivating group covalently bonded to a surface thereof.

26. The substrate of claim 20, wherein the substrate surface passivating group is further passivated by coating with albumin or gelatin.

27. The substrate of claim 20, wherein the surface tether comprises a substituted or unsubstituted C₃-C₃₀ alkyl residue or a polyethylene glycol residue.

28. The substrate of claim 20, wherein the surface tether comprises a polyethylene glycol residue having a molecular weight of from about 300 to about 10,000,000 Daltons.

29. The substrate of claim 28, wherein the polyethylene glycol residue has a molecular weight of about 2000 Daltons.

30. The substrate of claim 28, wherein the polyethylene glycol tethers of two or more receptors are linked by an amide bond.

31. The substrate of claim 20, wherein the cysteine is substituted with selenocysteine, methionine, or histodine.

32. A process for making a cysteine derivatized substrate, comprising: attaching one or more surface tethers comprising a cysteine residue to a surface of a substrate to provide a cysteine derivatized substrate; wherein the cysteine residue is capable of reacting with a receptor.

33. The process of claim 32, further comprising reacting the cysteine derivatized substrate with a receptor under conditions effective to form a covalent bond between the receptor and the cysteine residue.

34. The process of claim 32, wherein the substrate comprises glass, quartz, silicon dioxide wafers, or gold-coated surfaces.

35. The process of claim 32, wherein the cysteine residue is not directly covalently bonded to the surface of the substrate.

36. The process of claim 32, wherein the receptor is a protein, polypeptide, or peptide.

37. The process of claim 32, wherein the receptor is a peptide, polypeptide, or protein thioester.

38. The process of claim 32, wherein the receptor is a peptide, polypeptide, or protein azide.

39. The process of claim 32, further comprising passivating the surface of the substrate prior to reacting the cysteine derivatized substrate with the receptor.

40. The process of claim 39, wherein the substrate surface passivating group is further passivated by coating with albumin or gelatin.

41. The process of claim 39, wherein the substrate surface passivation is achieved by creating a polyethylene glycol monolayer on the surface.

42. The process of claim 39, wherein the substrate surface passivation is achieved by backfilling with succinimide esters to block unreacted amines.

43. The process of claim 32, wherein the one or more surface tethers comprise a substituted or unsubstituted C₃-C₃₀ alkyl residue or a polyethylene glycol residue.

44. The process of claim 32, wherein the surface tether comprises a polyethylene glycol residue having a molecular weight of from about 1,000 to about 20,000 Daltons.

45. The process of claim 32, wherein the polyethylene glycol residue has a molecular weight of about 2000 Daltons.

46. The substrate of claim 43, wherein the polyethylene glycol tethers of two or more receptors are linked by an amide bond.

47. The process of claim 32, wherein the cysteine residue is substituted with selenocystein, methionine, or histodine.

48. A substrate prepared by the process of claim 32.

49. A substrate comprising a surface tether having a cysteine residue and a spacer; wherein the cysteine residue is covalently bonded to the spacer, and the spacer is covalently bonded to a surface of the substrate; wherein the spacer comprises polyethylene glycol, substituted or unsubstituted C₁-C₃₀ alkyl, or a peptide linker.
